(19) United States
(12) Patent Application Publication
(10) Pub. No.: US 2006/0047246 A1
(43) Pub. Date: Mar. 2, 2006

(54) Catheter with retractable needle, and methods of use and manufacture

(75) Inventor: Robert Anders, Scottsdale, AZ (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(73) Assignee: 3FI Products LLC

(21) Appl. No.: 10/924,550

(22) Filed: Aug. 24, 2004

(51) Int. Cl. A61M 5/178

(52) U.S. Cl. 604/164.08

(57) ABSTRACT

In an embodiment, a catheter apparatus includes a catheter tube having a hollow catheter interior, a proximal catheter end, and a distal catheter end. A catheter connector is coupled to the catheter tube at the proximal catheter end. The apparatus further includes a needle having a hollow interior channel, a proximal needle end, and an insertion point. A needle hub is coupled to the needle at the proximal needle end. In an embodiment, the apparatus further includes a retraction mechanism to enable the insertion point to be retracted to a position inside the catheter apparatus.

### TECHNICAL FIELD

This invention relates generally to catheters and, in particular, to an intravenous catheter in which the insertion point is retractable, and methods of making and using embodiments of a catheter.

### BACKGROUND

Intravenous (IV) catheters may be used to administer fluids directly into a patient's vascular system. An IV catheter includes a flexible tube, which is attached at a proximal end to a catheter connector. A handheld placement device, which includes a sharp tip needle, is used to insert the IV catheter into a patient's vein. For an “over the needle” type of catheter, prior to insertion, a needle is positioned within the catheter so that the needle's tip extends slightly beyond the distal end of the catheter. The opposite end of the needle extends through the catheter connector and is connected to a needle hub.

To insert the catheter, a person (e.g., a health care worker) inserts the needle point through the patient's skin and into the patient's vein. Via the catheter connector, the person pushes the distal end of the catheter toward and beyond the needle point, thus locating the catheter's distal end within the vein. Once the catheter is positioned in this way, the person withdraws the needle by applying pressure to the patient's vein near the insertion site, and grasping and pulling the needle hub in a direction away from the insertion site. This removes the needle and needle hub from the catheter and catheter connector. An exposed portion of the catheter is taped to the patient's skin, and the catheter connector is attached to a source of fluid. The fluid then flows through the catheter into the patient's vein.

After a catheter needle has been used, it may include various, dangerous, blood-borne pathogens. Accordingly, a used catheter insertion needle is considered a contaminated bio-hazard. An inadvertent “stick” from a used needle may result in the exposed person contracting hepatitis, AIDS, or some other communicable disease. Therefore, strict regulations for the use and disposal of used needles exist, in order to reduce the likelihood that health care workers and others may inadvertently expose themselves to blood and other bodily substances that may be present on or within used needles.

However, even with strict regulations and extensive education, inadvertent needle sticks continue to occur at alarming rates. Needle sticks have been experienced by persons administering shots, drawing blood, inserting catheters, and performing other actions with needles. Further, an improperly disposed of, used needle may stick other persons long after the initial use of the needle has occurred. Many of these inadvertent needle sticks have exposed unintended victims to deadly diseases, resulting in a significant amount of deaths.

### BRIEF DESCRIPTION OF THE DRAWINGS

FIG. 1 illustrates a perspective view of a catheter device, in accordance with an embodiment of the inventive subject matter;

FIG. 2 illustrates a cross-sectional view of a catheter device, in accordance with an embodiment;

FIG. 3 illustrates an enlarged, cross sectional view of a catheter device retraction mechanism in a non-retracted position, in accordance with an embodiment;

FIG. 4 illustrates an enlarged, cross sectional view of the retraction mechanism of FIG. 3 in a retracted position, in accordance with an embodiment;

FIG. 5 illustrates a cross-sectional view of a catheter device in a retracted position, in accordance with an embodiment;

FIG. 6 illustrates a cross-sectional view of a catheter device in a retracted position, in accordance with another embodiment;

FIG. 7 illustrates a cross-sectional view of a catheter device in a retracted position, in accordance with still another embodiment;

FIG. 8 illustrates a perspective view of a first portion of a retraction mechanism, in accordance with an embodiment;

FIG. 9 illustrates a cross-sectional view of a second portion of a retraction mechanism, in accordance with an embodiment;

FIG. 10 illustrates a cross-sectional view of the second portion of the retraction mechanism of FIG. 9 along section lines 10-10, in accordance with an embodiment;

FIG. 11 illustrates a cross-sectional view of the second portion of the retraction mechanism of FIG. 9 along section lines 10-10, in accordance with another embodiment;

FIG. 12 illustrates a flowchart of a method for using a catheter with a retractable needle, in accordance with an embodiment;

FIG. 13 illustrates a cross-sectional view of a catheter device after insertion of the insertion point into a vein, in accordance with an embodiment;

FIG. 14 illustrates a cross-sectional view of the catheter of FIG. 13 after retraction of the needle into the distal catheter end, in accordance with an embodiment;

FIG. 15 illustrates a cross-sectional view of the catheter of FIG. 14 after further advancement of the catheter into a patient's vein, in accordance with an embodiment;

FIG. 16 illustrates a flowchart of a method for manufacturing a catheter having a retractable needle, in accordance with an embodiment; and

FIG. 17 illustrates a flowchart of a method for manufacturing a catheter having a retractable needle, in accordance with another embodiment.

### DESCRIPTION OF THE EMBODIMENTS

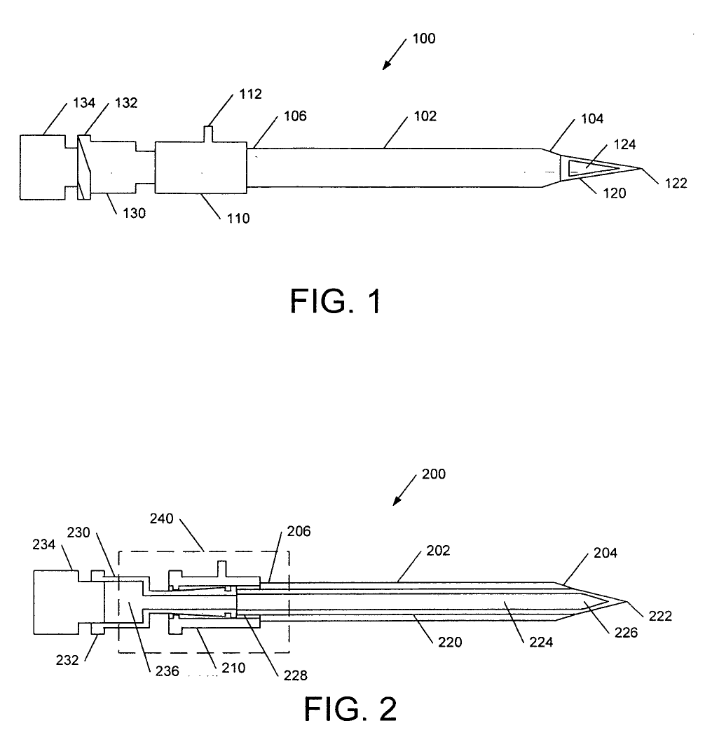

FIG. 1 illustrates a perspective view of a catheter device 100, in accordance with an embodiment of the inventive subject matter. Catheter device 100 may include, for example, an intravenous (IV) catheter, which is used to deliver fluids to a patient's vascular system. In other embodiments, catheter device 100 may be intended for other purposes, such as, for example, to drain fluids from a patient's body or to provide access to an area of the body (e.g., the chest cavity).

Catheter device 100 includes catheter tube 102, catheter connector 110, insertion needle 120, and needle hub 130. Further, in an embodiment, catheter device 100 includes a needle retraction mechanism, which may or may not be apparent from an exterior view of catheter device 100. The needle retraction mechanism is to enable the insertion point 122 of the needle 120 to be retracted to a position inside the catheter device (e.g., inside the catheter tube 102 and/or the catheter connector 110). Various embodiments of a needle retraction mechanism will be described in more detail later.

Catheter tube 102 has a hollow catheter interior (not depicted in FIG. 1), a distal catheter end 104, and a proximal catheter end 106. The hollow catheter interior extends between proximal catheter end 106 and distal catheter end 104, providing for the flow of fluids between and through openings at the two ends. In an embodiment, proximal catheter end 106 is coupled to a catheter connector 110. In an embodiment, catheter connector 110 includes an extension 112 or ridge, which facilitates advancement of catheter tube 102 in relation to insertion needle 120.

Insertion needle 120 is formed from a rigid material, such as stainless steel, in an embodiment. In an alternate embodiment, insertion needle 120 or its point are formed from a hardened, substantially soluble material. Prior to initial use of the catheter device 100, an insertion point 122 of the needle 120 extends beyond the distal catheter end 104. In an embodiment, the needle includes an internal channel (not depicted in FIG. 1), which has an opening 124 in proximity to the insertion point 122. The internal channel extends from opening 124 in a direction coaxial with the hollow catheter interior.

The needle is attached at a proximal needle end to a substantially hollow needle hub 130. In an embodiment, needle hub 130 includes a connector portion 132, to enable the needle hub to be connected to a fluid-delivery tubing connector (not depicted in FIG. 1). Prior to use, an end cap 134 may substantially seal an interior blood collection chamber (not depicted in FIG. 1) of the needle hub 130.

FIG. 2 illustrates a cross-sectional view of a catheter device 200, in accordance with an embodiment. In an embodiment, the catheter device 200 includes catheter tube 202, catheter connector 210, insertion needle 220, needle hub 230, and a partial needle retraction mechanism. In an embodiment, the partial needle retraction mechanism is located in an area 240 proximate to catheter connector 210 and needle hub 230.

Catheter tube 202 includes a hollow catheter interior, a distal catheter end 204, and a proximal catheter end 206. In an embodiment, proximal catheter end 206 is coupled to a catheter connector 210.

Insertion needle 220 includes an insertion point 222 and a hollow needle channel 224. Channel 224 has an opening 226 in proximity to insertion point 222, and channel 224 extends in a direction coaxial to the hollow catheter interior. Prior to initial use of the catheter device 200, insertion point 222 extends beyond the distal catheter end 204.

In an embodiment, a proximal needle end 228 is coupled to a substantially hollow needle hub 230. In an embodiment, needle hub 230 includes a connector portion 232 (e.g., a threaded portion), which provides for attachment of the needle hub 230 to fluid-delivery tubing after the distal catheter end 204 has been inserted at the intended location (e.g., into a patient's vein). In an alternate embodiment, needle hub 230 may be configured so that fluid-delivery tubing may fit over needle hub 230 and connect instead to the catheter connector 210.

An end cap 234 may substantially seal an interior blood collection chamber 236 of the needle hub 230. During use, if the insertion point 222 is properly inserted into a vein, a small amount of blood may flow into needle opening 226, through the needle channel 224, and into the blood collection chamber 236. In an embodiment, the blood collection chamber 236 is formed from a material that enables the catheter user to observe blood in the blood collection chamber 236.

The catheter device 200 depicted in FIG. 2 is in a “non-retracted” position. In such a position, insertion point 222 extends outside of and beyond distal catheter end 204. In an embodiment, after the distal catheter end 204 has been properly inserted (e.g., into a vein), the insertion point 222 is partially retracted so that it does not extend beyond the distal catheter end 204. The catheter tube 202 may then be advanced further into the vein.

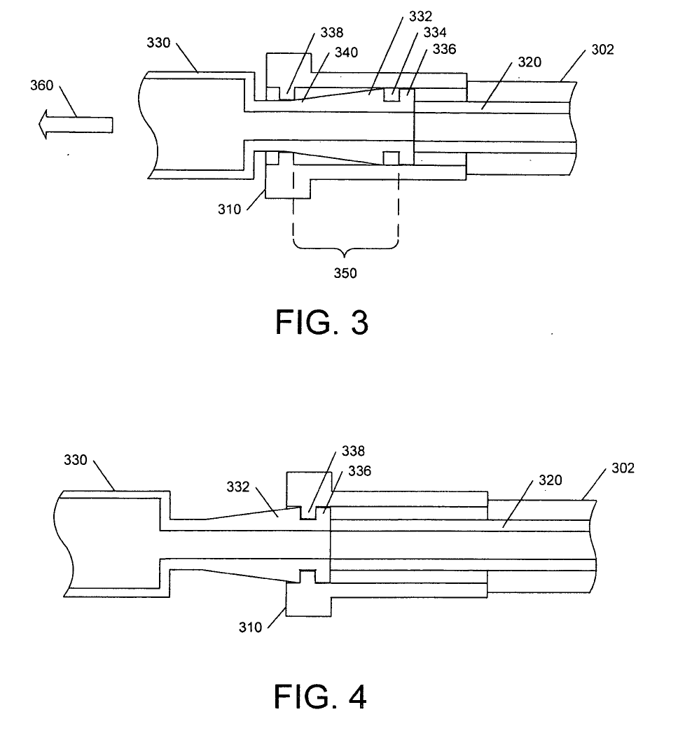

FIG. 3 illustrates an enlarged, cross sectional view of an area (e.g., area 240, FIG. 2) of a catheter device retraction mechanism in a non-retracted position, in accordance with an embodiment. In an embodiment, the retraction mechanism includes complementary mechanical mechanisms associated with catheter connector 310 and needle hub 330. In an embodiment, needle hub 330 includes one or more angled glides 332, one or more notches 334, and one or more first glide stop mechanisms 336. Catheter connector 310 includes one or more second glide stop mechanisms 338, which may be shaped to be engagable with notches 334. In an alternate embodiment, catheter connector 310 includes one or more glides, notches, and first glide stop mechanisms, and needle hub 330 includes one or more second glide stop mechanisms. In other words, in an alternate embodiment, portions of a partial retraction mechanism may be located on opposite ones of catheter connector 310 and needle hub 330 than is illustrated in FIG. 3.

In a non-retracted position, first and second glide stop mechanisms 336, 338 are respectively positioned so that second glide stop mechanisms 338 are proximate to a first section 340 of angled glides 332, where the first section 340 is located approximately a retraction distance 350 from notches 334 and/or first glide stop mechanisms 336.

To initiate partial retraction of a needle 320 attached to needle hub 330, with respect to a catheter tube 302, a person may pull needle hub 330 in a direction indicated by arrow 360. The pulling force causes notches 334 to move in a direction toward second glide stop mechanisms 338, while second glide stop mechanisms 338 slideably engage angled glides 332. Eventually, notches 334 will reach second glide stop mechanisms 338, and second glide stop mechanisms 338 will engage with notches 334, placing the retraction mechanism in a retracted position. Further movement of second glide stop mechanisms 338 with respect to notches 334 is limited by substantially parallel sides of angled glides 332 and first glide stop mechanisms 336.

FIG. 4 illustrates an enlarged, cross sectional view of the retraction mechanism of FIG. 3 in a retracted position, in accordance with an embodiment. As illustrated in FIG. 4, second glide stop mechanisms 338 of catheter connector 310 are engaged with notches of the needle hub 330 (i.e., notches 334, FIG. 3). Further movement of second glide stop mechanisms 338 with respect to the notches is limited by substantially parallel sides of angled glides 332 and first glide stop mechanisms 336. The at least partial retraction of needle 320 with respect to catheter tube 302 also results in the at least partial retraction of the insertion point (e.g., point 222, FIG. 2) with respect to the distal end of the catheter tube 302 (e.g., distal end 204, FIG. 2).

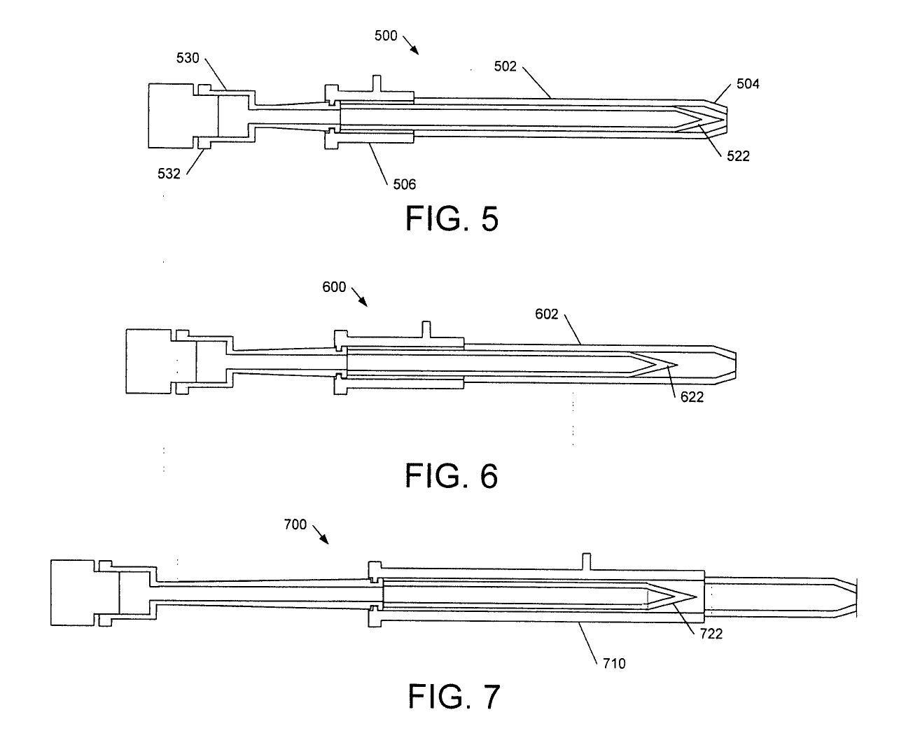

FIG. 5 illustrates a cross-sectional view of a catheter device 500 in a retracted position, in accordance with an embodiment. In the retracted position, second glide stop mechanisms (e.g., mechanisms 338, FIG. 4) of catheter connector 506 are held in place with respect to notches by substantially parallel sides of angled glides (e.g., glides 332, FIG. 4) and first glide stop mechanisms (e.g., mechanisms 336, FIG. 4) of needle hub 530.

In this position, insertion point 522 is retracted past distal catheter end 504 to a position within catheter tube 502. Although insertion point 522 is illustrated to be retracted to a position just inside distal catheter end 504, in other embodiments, the retraction distance may be longer, thus resulting in insertion point 522 being retractable to a position further inside catheter tube 502. As mentioned previously, needle hub 530 may include a connector 532 to facilitate attachment of fluid-delivery tubing.

FIG. 6 illustrates a cross-sectional view of a catheter device 600 in a retracted position, in accordance with another embodiment. In the embodiment illustrated in FIG. 6, the retraction mechanism is elongated, providing for retraction of insertion point 622 to a position further inside catheter tube 602.

FIG. 7 illustrates a cross-sectional view of a catheter device 700 in a retracted position, in accordance with still another embodiment. In the embodiment illustrated in FIG. 7, the retraction mechanism is elongated further yet, providing for retraction of insertion point 722 to a position inside catheter connector 710.

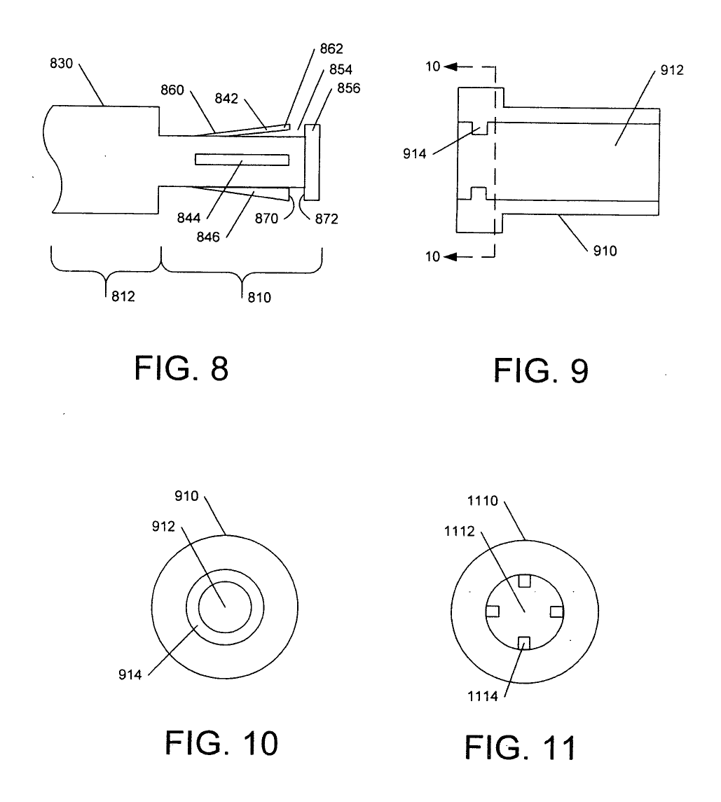

In an embodiment, the needle retraction mechanism includes at least two portions, where a first portion is connected to the needle hub, and the second portion is connected to the catheter connector. FIG. 8 illustrates a perspective view of a first portion of a retraction mechanism, in accordance with an embodiment. The first retraction mechanism portion is integrally attached to needle hub 830, in an embodiment. In an alternate embodiment, the first portion is otherwise connected to the needle hub. Needle hub 830 generally includes a shaft section 810 and a blood collection chamber portion 812 (only part of the blood collection chamber portion is illustrated in FIG. 8).

On the shaft section 810 of needle hub 830, the first retraction mechanism portion includes one or more angled glides 842, 844, 846, one or more slots 854, and one or more retraction stop mechanisms 856, in an embodiment. Although three angled glides are illustrated in FIG. 8, the first retraction mechanism may include as few as one angled glide or several (e.g., four or more) angled glides distributed around the circumference of the shaft 810.

In an embodiment, one or more angled glides (e.g., glide 842) may include an elongated member, which is attached to shaft 810 at a first end 860, and free at a second end 862. A space may exist between the second end 862 and an adjacent portion of the shaft 810, allowing for deflection of the second end 862 toward shaft 810 as the catheter connector's retraction mechanism (e.g., mechanism 338, FIG. 3) slides along the elongated member during needle retraction. In an embodiment, the elongated member has a sufficient spring coefficient to enable it to deflect toward shaft 810, and then snap back toward its original, undeflected position, after the catheter connector's retraction mechanism has slid into slot 854.

In another embodiment, one or more angled glides (e.g., glide 846) may include a wedge-shaped member, which is in contact with shaft 810 along a substantial portion of the glide's length. Once the catheter connector's retraction mechanism has slid into slot 854, it is held in place by substantially parallel sides 870, 872 of angled glides 842, 844, 846 and retraction stop mechanism 856.

FIG. 9 illustrates a cross-sectional view of a second portion of a retraction mechanism, in accordance with an embodiment. The second retraction mechanism portion is integrally attached to catheter connector 910, in an embodiment. In an alternate embodiment, the second portion is otherwise connected to the catheter connector.

The second retraction mechanism portion includes one or more retraction stop mechanisms 914, in an embodiment, which extend into an interior channel 912 of catheter connector 910. As described previously, retraction stop mechanisms 914 slideably engage one or more of a needle hub's angled glides (e.g., glides 842, 844, 846, FIG. 8), and engage one or more needle hub slots (e.g., slot 854, FIG. 8). In an embodiment, retraction stop mechanism 914 includes a ring, as will be illustrated and described in conjunction with FIG. 10. In another embodiment, retraction stop mechanism 914 includes one or more tabs, as will be described in conjunction with FIG. 11.

FIG. 10 illustrates a cross-sectional view of the second portion of a retraction mechanism of FIG. 9 along section lines 10-10, in accordance with an embodiment. The second portion of the retraction mechanism includes a ring 914, which extend inward into the interior channel 912 of catheter hub 910.

FIG. 11 illustrates a cross-sectional view of the second portion of a retraction mechanism of FIG. 9 along section lines 10-10, in accordance with another embodiment. The second portion of the retraction mechanism includes one or more tabs 1114, which extend inward into the interior channel 1112 of catheter hub 1110.

Besides using a retraction mechanism having substantially a same design as the retraction mechanisms illustrated in conjunction with FIGS. 2-11, other types of retraction mechanisms alternatively could be used, as may be apparent to those of skill in the art based on the description herein. Numerous types of retraction mechanisms may be used to achieve the same purpose of retracting the insertion point into the catheter.

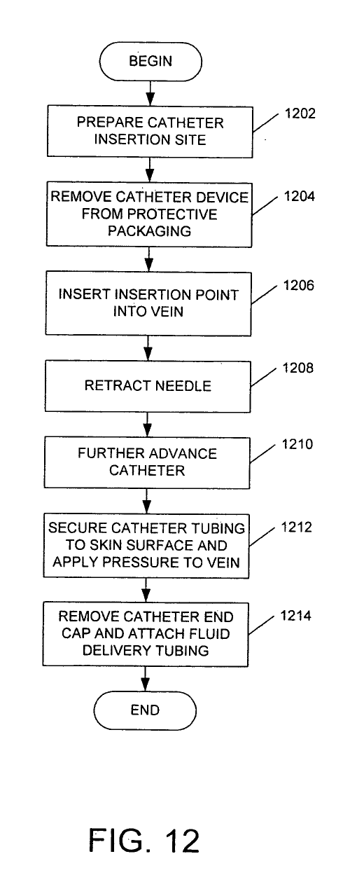

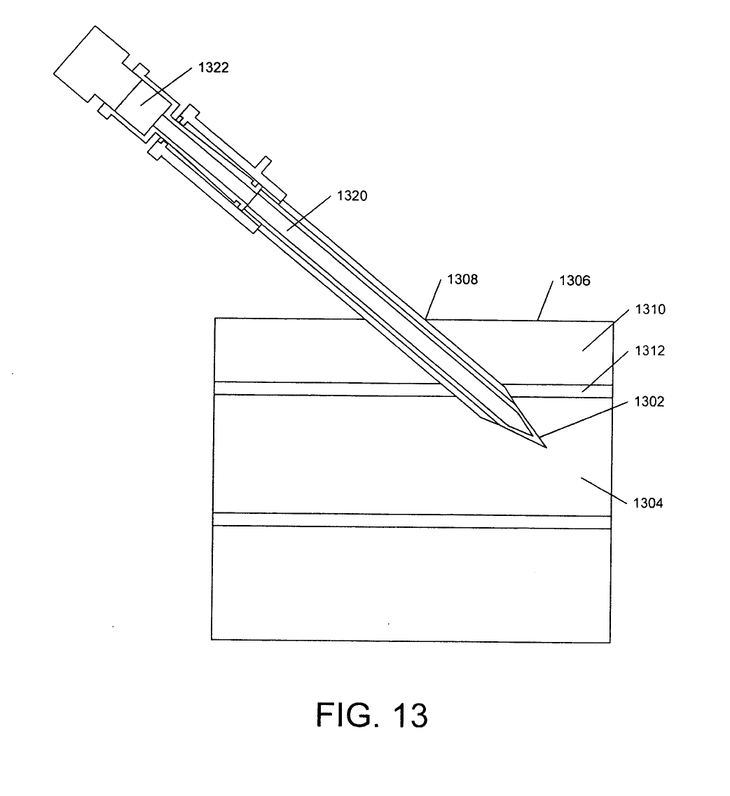

FIG. 12 illustrates a flowchart of a method for using a catheter with a retractable insertion point, in accordance with an embodiment. The method begins, in block 1202, by preparing a catheter insertion site on a body. This may include, for example, a person visually locating a candidate vein by inspection of the surface of a patient's skin, and cleaning the skin overlying the vein with an antibacterial solution or disinfectant (e.g., iodine and/or alcohol).

In block 1204, the catheter device (i.e., the assembled catheter, catheter hub, needle, needle hub, and end cap) is removed from protective packaging. In an embodiment, the packaging is used to ensure that the catheter device remains sterile prior to use.

In block 1206, the insertion point is inserted at the insertion site through the surface of the skin and into the patient's vein. FIG. 13 illustrates a cross-sectional view of a catheter after insertion of the insertion point 1302 into an interior channel of a vein 1304, in accordance with an embodiment. The insertion point 1302 is inserted through the surface 1306 of the patient's skin at an insertion site 1308. The insertion point 1302 is then advanced through any intermediate tissue 1310, through the vein's top lumen 1312, and into the interior channel 1304. When so inserted, blood located in the interior channel 1304 may flow up through the needle channel 1320 and into blood collection chamber 1322. Proper insertion into a vein may be verified by observing the blood in the blood collection chamber 1322.

Referring back to FIG. 12, the needle is retracted in block 1208. In an embodiment, the needle is partially retracted, as facilitated by the use of a partial retraction mechanism. Retraction is achieved by manipulating the needle hub with respect to the catheter connector so that the retraction mechanism transitions from a non-retracted position to a retracted position.

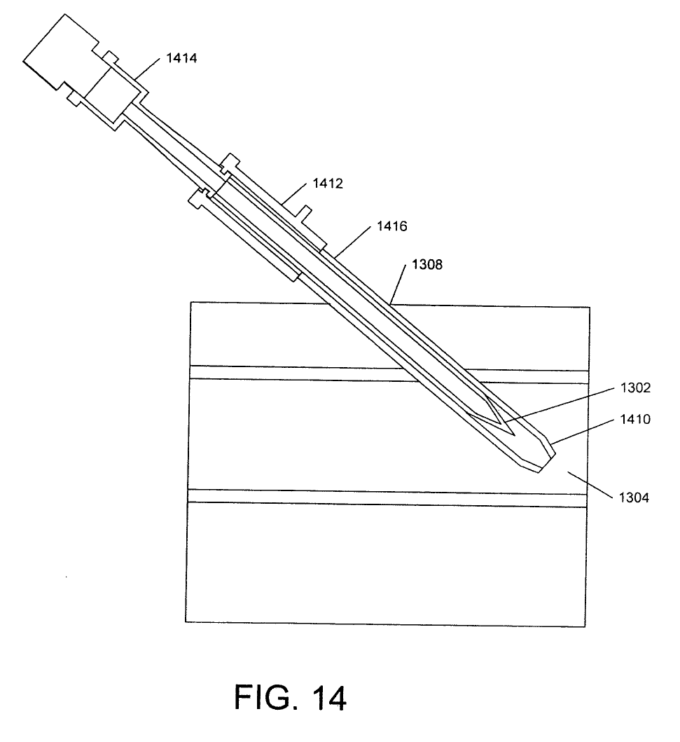

FIG. 14 illustrates a cross-sectional view of the catheter of FIG. 14 after retraction of the insertion point 1302 into the distal catheter end 1410, in accordance with an embodiment. As described previously, the needle may be partially retracted by holding the catheter connector 1412 stationary with respect to the insertion site 1308, and pulling the needle hub 1414 outwardly away from the body. Alternatively, the needle hub 1414 may be held stationary with respect to the insertion site 1308, and the catheter connector 1412 may be pushed toward the insertion site 1308, thus advancing the distal catheter end 1410 beyond the insertion point 1302 and further into the interior channel 1304 of the vein. Either way, once partially retracted, the insertion point 1302 is positioned inside the catheter tube 1416, in an embodiment. In another embodiment, the insertion point may be retracted all the way out of the catheter tube, and into the catheter connector.

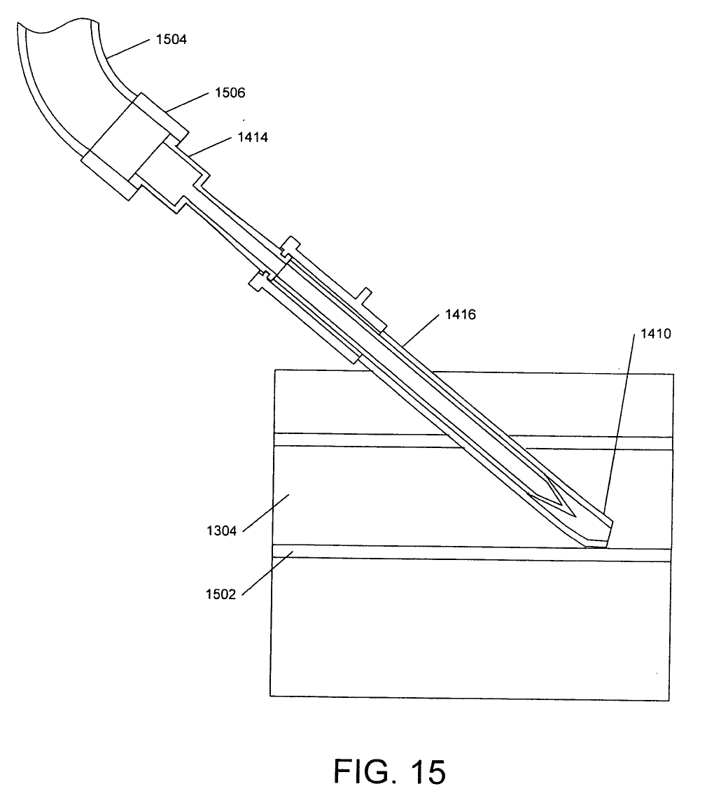

Referring again to FIG. 12, in block 1210, the distal catheter end is advanced further into the patient's vein. FIG. 15 illustrates a cross-sectional view of the catheter of FIG. 14 after further advancement of the catheter into a patient's vein, in accordance with an embodiment. During advancement, the distal catheter end 1410 may contact a bottom side of the vein's lumen 1502. However, for normal veins, the distal catheter end 1410 will lack sufficient sharpness to puncture the lumen 1502, in an embodiment. Accordingly, the catheter tube 1416 may bend while being advanced further into the interior channel of the vein 1304.

Referring again to FIG. 12, in block 1212, the catheter connector and/or tube are secured to the surface of the patient's skin using medical tape, for example, and pressure is applied to the patient's vein in proximity to the insertion site, in order to stem the flow of blood through the vein. The catheter end cap is removed, in block 1214, and fluid-delivery tubing is attached to the device. Referring again to FIG. 15, fluid-delivery tubing 1504 is shown to be attached, via tubing connector 1506, to needle hub 1414. In an embodiment, the needle hub 1414 is designed to attach to the fluid-delivery tubing connector 1506 using a threaded connection. In another embodiment, the tubing connector fits over the needle hub and is attachable to the catheter connector.

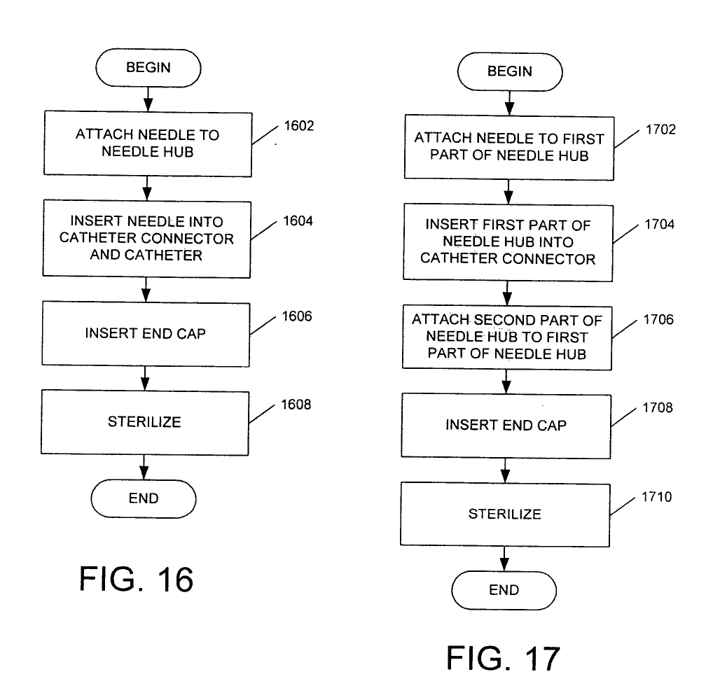

A method for manufacturing a catheter device, in accordance with various embodiments, basically involves assembling a catheter tube, a catheter connector, a needle, and a needle hub. FIG. 16 illustrates a flowchart of a method for manufacturing a catheter device having a retractable insertion needle, in accordance with an embodiment. The method begins, in block 1602, by attaching a needle to a needle hub. In an embodiment, the needle hub includes a first portion of a retraction mechanism (e.g., as illustrated in FIG. 7).

In block 1604, the needle is inserted through the catheter connector and catheter tube so that the insertion point extends beyond the distal end of the catheter tube. In another embodiment, the catheter connector may be snapped or otherwise fastened around the needle hub, and the catheter tube may be slid over the needle and attached to the catheter connector.

In block 1606, the end cap is inserted into the blood collection chamber of the needle hub. The catheter device is then sterilized, in block 1608. In an embodiment, sterilization may include irradiating the catheter device (e.g., by gamma or E-beam irradiation) or by exposure to a gaseous sterilization agent (e.g., ethylene oxide gas). These processes may be carried out at controlled temperatures and humidity conditions. After sterilization, the catheter device is ready for packaging. The method then ends.

In various embodiments, the catheter connector and needle hub may not be readily slid together during manufacture, due to the various portions of the catheter connector and needle hub. Accordingly, in several embodiments, the needle hub, the catheter connector, or both may be formed from multiple parts, which may be connected together during manufacture of the catheter device.

FIG. 17 illustrates a flowchart of a method for manufacturing a catheter having a retractable insertion needle, in accordance with another embodiment. In the illustrated embodiment, the needle hub includes at least two parts, which are separable, but are designed to be connected during manufacture, as described below. For example, the needle hub may include a shaft part (e.g., part 810, FIG. 8) and a blood collection chamber part (e.g., part 812, FIG. 8).

A method for fabricating a catheter device having such a structure begins, in block 1702, by attaching a needle to a first part (e.g., shaft 810, FIG. 8) of the needle hub. In an embodiment, the first part of the needle hub includes a hollow shaft with an exterior diameter that is small enough to slide into the interior channel of the catheter connector. The hollow shaft further includes one or more angled glides, notches, and first glide stop mechanisms.

In block 1704, the first part of the needle hub is inserted into the catheter connector. In an embodiment, the catheter tube is already attached to the catheter connector, and so the needle hub and needle are also inserted through the catheter tube so that the insertion point extends beyond the distal end of the catheter tube. In another embodiment, the catheter tube may be slid over the needle and attached to the catheter connector after the first part of the needle hub is slid into the catheter connector.

In block 1706, a second part (e.g., blood collection chamber part 812, FIG. 8) of the needle hub is attached to the first part of the needle hub. In an embodiment, the second part of the needle hub includes the blood collection chamber, and may have an exterior diameter that is greater than the interior diameter of the catheter connector.

In block 1708, an end cap is inserted into the blood collection chamber of the needle hub. The catheter device is then sterilized, in block 1710, packaged, and the method ends.

The various blocks depicted in FIGS. 12, 16, and 17 may be performed in different orders from the orders illustrated and described, while still achieving the same results. Additionally, some blocks may be performed in parallel, rather than sequentially.

Although specific embodiments have been illustrated and described herein, it will be appreciated by those of ordinary skill in the art that any arrangement that is calculated to achieve the same purpose may be substituted for the specific embodiments shown. In particular, retraction mechanisms having different configurations from the specific embodiments described may be used. For example, but not by way of limitation, an alternate retraction mechanism may include complementary threaded members associated with a catheter connector and a needle hub, where retraction is accomplished by twisting the catheter connector with respect to the needle hub.

Adaptations of the invention may be apparent to those of ordinary skill in the art, based on the description herein. Accordingly, this application is intended to cover apparent adaptations or variations of the inventive subject mater. It is manifestly intended that this invention be limited only by the following claims and equivalents thereof.

What is claimed is:

1. A catheter apparatus comprising:
a catheter tube having a hollow catheter interior, a proximal catheter end, and a distal catheter end;
a catheter connector, coupled to the catheter tube at the proximal catheter end;
a needle having a hollow interior channel, a proximal needle end, and an insertion point;
a needle hub, coupled to the needle at the proximal needle end; and
a retraction mechanism to enable the insertion point to be retracted to a position inside the catheter apparatus.

2. The catheter apparatus of claim 1, wherein the insertion point extends beyond the distal catheter end when the retraction mechanism is in a non-retracted position, and the insertion point is located within the catheter apparatus when the retraction mechanism is in a retracted position.

3. The catheter apparatus of claim 1, wherein the retraction mechanism is to enable the insertion point to be retracted to a position inside the catheter tube.

4. The catheter apparatus of claim 1, wherein the retraction mechanism is to enable the insertion point to be retracted to a position inside the catheter connector.

5. The catheter apparatus of claim 1, wherein the retraction mechanism comprises:
a first portion of the retraction mechanism coupled to the needle hub; and
a second portion of the retraction mechanism coupled to the catheter connector.

6. The catheter apparatus of claim 1, wherein the retraction mechanism comprises:
one or more angled glides and one or more first glide stop mechanisms coupled to the needle hub; and
one or more second glide stop mechanisms coupled to the catheter connector, wherein the one or more second glide stop mechanisms are configured to engage one or more notches defined by the one or more angled glides and the one or more first glide stop mechanisms.

7. The catheter apparatus of claim 1, wherein the retraction mechanism comprises:
one or more angled glides and one or more first glide stop mechanisms coupled to the catheter connector; and
one or more second glide stop mechanisms coupled to the needle hub, wherein the one or more second glide stop mechanisms are configured to engage one or more notches defined by the one or more angled glides and the one or more first glide stop mechanisms.

8. The catheter apparatus of claim 1, wherein the retraction mechanism comprises:
complementary threaded members associated with the catheter connector and the needle hub.

9. The catheter apparatus of claim 1, wherein the needle hub comprises:
a connector portion to attach the catheter apparatus to fluid-delivery tubing.

10. A method for using a catheter apparatus, the method comprising:
inserting an insertion point of a catheter needle into a body, wherein the insertion point extends beyond a distal end of a catheter tube; and
retracting the insertion point into the distal end of the catheter tube.

11. The method of claim 10, wherein retracting the insertion point comprises:
retracting the insertion point to a retracted position within the catheter tube.

12. The method of claim 10, wherein retracting the insertion point comprises:
retracting the insertion point to a retracted position within a catheter connector.

13. The method of claim 10, wherein retracting the insertion point comprises:
manipulating a needle hub coupled to the catheter needle with respect to a catheter connector coupled to the catheter tube so that a retraction mechanism, coupled to the needle hub and the catheter connector, transitions from a non-retracted position to a retracted position.

14. A method for making a catheter apparatus, the method comprising:
assembling a catheter tube, a catheter connector, a needle, and a needle hub, wherein the catheter tube and the needle hub include a needle retraction mechanism, and wherein assembling the catheter tube includes inserting the needle into the catheter tube so that an insertion point of the needle extends beyond a distal end of the catheter tube, when the needle retraction mechanism is in a non-retracted position.

15. The method of claim 14, wherein assembling further comprises:
assembling the needle hub and the catheter connector, wherein the needle hub includes one or more angled glides and one or more first glide stop mechanisms coupled to the needle hub.

16. The method of claim 15, wherein assembling further comprises:
assembling the needle hub and the catheter connector, wherein the catheter connector includes one or more second glide stop mechanisms coupled to the catheter connector, wherein the one or more second glide stop mechanisms are configured to engage one or more notches defined by the one or more angled glides and the one or more first glide stop mechanisms.

17. The method of claim 14, wherein assembling further comprises:
assembling the needle hub and the catheter connector, wherein the needle hub includes one or more second glide stop mechanisms coupled to the needle hub, wherein the one or more second glide stop mechanisms are configured to engage one or more notches defined by the one or more angled glides and the one or more first glide stop mechanisms.
